(19) United States
(12) Patent Application Publication
(10) Pub. No.: US 2021/0129657 A1
(43) Pub. Date: May 6, 2021

(54) TENSEGRITY STRUCTURES WITH FORCE-BASED MOTION AND APPLICATIONS THEREOF

(71) Applicant: THE UNIVERSITY OF NORTH CAROLINA AT CHAPEL HILL, Chapel Hill, NC (US)

(72) Inventor: Henry Paul GOODELL, Raleigh, NC (US)

(21) Appl. No.: 17/046,363

(22) PCT Filed: Apr. 10, 2019
(86) PCT No.: PCT/US2019/026826

§ 371 Date: Oct. 9, 2020

Related U.S. Application Data
(60) Provisional application No. 62/655,543, filed on Apr. 10, 2018.

(51) Int. Cl. B60K 7/00; B64G 1/10; G06F 30/20

(52) U.S. Cl. CPC B60K 7/0007; B64G 1/10; A61B 5/1123; B60K 2007/0092; G06F 30/20

(57) ABSTRACT

In one aspect, tensegrity structures or apparatus are described herein comprising rings or hoops. Briefly, a tensegrity structure comprises a plurality of rings spaced apart along a central axis and connected by struts, wherein the struts are coupled to a force actuator assembly via cables, the force actuator assembly residing within a volume defined by the struts. In some embodiments, the tensegrity structure employs three struts for connecting the rings, although any desired number of struts is possible. Moreover, cables can extend from opposing ends of the force actuator assembly to couple with the struts.

### RELATED APPLICATION DATA

The present application claims priority pursuant to 35 U.S.C. § 119(e) to U.S. Provisional Patent Application Ser. No. 62/655,543 filed Apr. 10, 2018 which is incorporated herein by reference in its entirety.

### FIELD

The present invention relates to tensegrity structures and assemblies and, in particular, to tensegrity structures comprising rings or hoops connected by struts.

### BACKGROUND

Previous tensegrity robotic structures for surface exploration are fundamental tensegrity structures of pure rods and cables. These structures provide central load impact protection, while enabling a rolling-type motion for surface exploration. Numerous independent cables with a distributed control scheme may present challenges, and the various rods create possible point impact areas at their ends. These points may be beneficial in circumstances of low gravity or soft surfaces when anchoring could be beneficial. Taken together, these challenges, however solvable, may hinder their implementation and acceptance.

Previous tensegrity wheel designs have incorporated actuated joints to change the radius of the wheel for moment-shifting-induced rolling. This generalized design, while maintaining a single central area to house sensors and motors, may expose joints to a dusty or granular surface, possibly degrading their performance. The folded storage of these designs demonstrates the potential for efficient packing of a tensegrity structure for launch.

### SUMMARY

In one aspect, tensegrity structures and assemblies are described herein comprising rings or hoops. Briefly, a tensegrity structure comprises a plurality of rings spaced apart along a central axis and connected by struts, wherein the struts are coupled to a force actuator assembly via cables, the force actuator assembly residing within a volume defined by the struts. In some embodiments, the tensegrity structure employs three struts for connecting the rings, although any desired number of struts is possible. Moreover, cables can extend from opposing ends of the force actuator assembly to couple with the struts. The cables, for example, can couple to the struts in pairs, one set of cables originating from each end of the force actuator assembly. Additionally, the force actuator assembly can reside outside the volume defined by the rings, depending on the desired force or movement condition during operation of the tensegrity structure.

In another aspect, a tensegrity structure comprises a cylinder extending along a central axis and connected to a plurality of struts, wherein the struts are coupled to a force actuator assembly via cables, the force actuator assembly residing within a volume defined by the struts. As described above, the tensegrity structure may comprise three struts coupled to the cylinder, wherein cables originate from both ends of the force actuator assembly. Depending on the force condition, the force actuator assembly may reside outside the volume defined by the cylinder, in some embodiments.

Tensegrity structures and apparatus described herein can be used in a variety of applications. In some embodiments, the tensegrity structure serves as a wheel. Forward/backward rolling motion is accomplished through actuating the cables to shift the force actuator's center of mass around the circumference of the rings or cylinder in either direction. Left/right steering while rotating is accomplished by offsetting the actuator's center of mass axially to one side of the wheel. In such embodiments, the force actuator can serve as an axle for the wheel.

To limit reliance on encoders, a cable force and accelerometer control scheme can be employed. Cable tension and accelerometer data may be utilized to determine the axle's center of mass in relation to gravity and motion. Additionally the cable tension may be compared to the angular force from the force sensor furthest from the motor to determine an approximate angle for localization of the central axle. Additionally the AC components from the tension or angular force sensors can be isolated and when compared to the tension may be used for cable length determination. Rolling distance traveled is encoded through axle rotations.

Tensegrity structures described herein can also find application in communication and telescope systems, rotating cylinder kite apparatus and thrust vectoring systems and apparatus. These applications are further detailed in the following detailed description.

As described above, tensegrity structures of the present application can provide simultaneous rotational and axial movements through force measurements alone. The tensegrity structures can also provide non-linear motion profiles that more closely mimic human movements, such as human running and/or walking motion dynamics. Accordingly, tensegrity structures can be employed in studying human and/or animal motion. A tensegrity structure can serve as a robot or simulator for inducing motion in a model of the human body or a model of any portion of the human body. In some embodiments, for example, the tensegrity structure can be coupled to a model of the human torso for studying running and/or walking motions. Data generated during the study can be used for various applications, including elucidation of how various clothing articles perform relative to human movement. Rheological and/or other properties of clothing articles can be accurately examined in this way.

These and other embodiments are further described in the following detailed description.

### BRIEF DESCRIPTION OF THE DRAWINGS

FIGS. 1A-E illustrate various views of a converging axle wheel configuration of a tensegrity structure according to some embodiments.

FIGS. 2A-G illustrate various views of a diverging central mass wheel configuration of a tensegrity structure having flexing struts according to some embodiments.

FIGS. 3A-C illustrate rotational movement of a wheel tensegrity structure according to some embodiments.

FIGS. 4A-C illustrate angular force and tension sensor configurations at axle ends according to some embodiments.

FIG. 5 illustrates converging axle and PEX rim builds of a tensegrity structure according to some embodiments.

FIG. 6 illustrates a polypropylene tubing rim build of a tensegrity structure according to some embodiments.

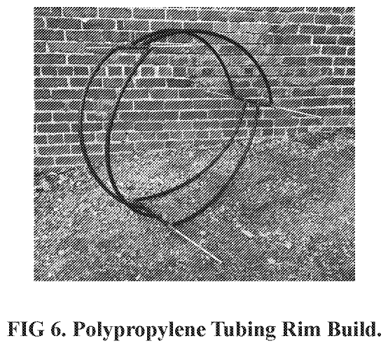

FIG. 7 illustrates angular force to tension ratio versus cable angle of a tensegrity structure according to some embodiments.

FIG. 8 illustrates an embodiment of a force actuator assembly serving as an axle for the tensegrity structure according to some embodiments.

FIG. 9 illustrates a perspective view of a tensegrity structure comprising the force activator assembly of FIG. 8, according to some embodiments.

FIG. 10 illustrates a perspective view of a tensegrity structure employing a hollow rotational motor attached to a central axle, according to some embodiments.

FIG. 11 is a magnified view of the tensegrity structure of FIG. 10.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In one aspect, a tensegrity structure comprises a plurality of rings spaced apart along a central axis and connected by struts, wherein the struts are coupled to a force actuator assembly via cables, the force actuator assembly residing within a volume defined by the struts. In some embodiments, the tensegrity structure employs three struts for connecting the rings, although any desired number of struts is possible. Moreover, cables can extend from opposing ends of the force actuator assembly to couple with the struts. The cables, for example, can couple to the struts in pairs, one set of cables originating from each end of the force actuator assembly. Additionally, the force actuator assembly can reside outside the volume defined by the rings, depending on the desired force or movement condition during operation of the tensegrity structure.

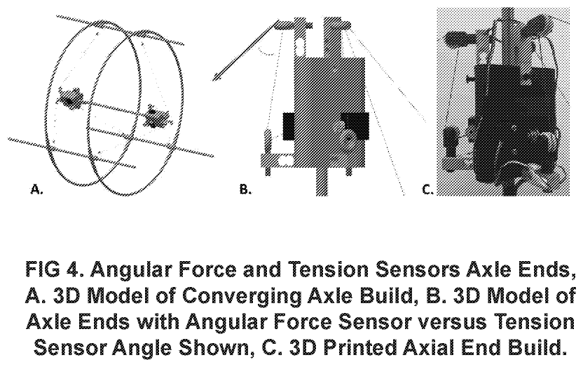

Turning now to specific components, the rings can have any desired dimensions and shape consistent with the objectives of the present invention. In some embodiments, the rings are circular or elliptical. Circular or elliptical rings, for example, can be employed in applications wherein the tensegrity structure laterally and/or vertically translates via rotation of the rings. Alternatively, the rings may be polygonal, in some embodiments. Polygonal rings may be used for stationary applications of the tensegrity structure, in some embodiments. The rings are spaced apart along a central axis and connected to struts, wherein the struts are coupled to a force actuator assembly via cables, the force actuator assembly residing within a volume defined by the struts. In some embodiments, the force actuator assembly can serve as the central axle of the tensegrity structure. The central axle can contain all of the electronics, motors, sensors, batteries, and/or instruments, in some embodiments. In one design-build, no traditional encoders on the cables or motors, absolute or incremental, are included. Instead, forces on the cables, both angular and tension, are used for locomotion and control. An angular force to tension sensor producing angle data for triangulation can be expanded to include all sensor motifs: angular, absolute, and incremental sensors. Combining triangulation and trilateration minimizes error across the workspace of the tensegrity structure and any associated robotic apparatus. FIGS. 4A-C illustrate angular force and tension sensor configurations at axle ends according to some embodiments.

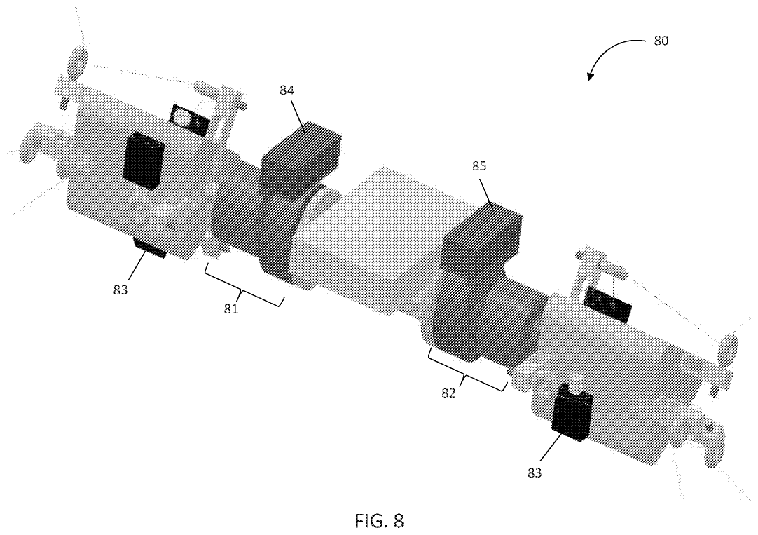

FIG. 8 illustrates another embodiment of a force actuator assembly serving as an axle for the tensegrity structure. The force actuator assembly of FIG. 8 can comprise one or a plurality of slip rings adjoined to hollow servomotors. In the embodiment of FIG. 8, two servo slip rings 81, 82 are part of the force actuator assembly 80. The two servo slip rings 81, 82 are centrally located on the assembly 80. In other embodiments, the servo slip rings may be distal along the axle. In the embodiment of FIG. 8, distal ends of the force actuator 80 comprise cable motor/encoders 83 with angular force and tension sensors as described further in relation to FIG. 4.

Onto the servo slip rings 81/82, batteries and/or other weighted materials 84, 85 to be transported by the tensegrity structure may be attached. The batteries 84, 85 can be distributed around the servo slip ring evenly or be localized at a point around the slip ring rotational axis. Movement of the two gyroscopic slip ring masses 81/84, 82/85 provides an additional torque aspect to motion of the tensegrity structure, both in rolling and positional change.

When the servo slip rings rotate in the same direction, the moment of inertia is maximized, and torque and inertia are transferred through the cables to one or more of the rings. When the servo slip rings counter-rotate equally, their gyroscopic forces cancel out. Inertial and torque-driven control may be accomplished through these mechanisms, increasing efficiency of rolling and other motions. Sensors and controls may be placed on any part of the axle and/or servo slip ring(s). 3-dimensional scanning on the slip ring (verses the rotating axle), may provide front/rear facing view or other ground-references view while rolling.

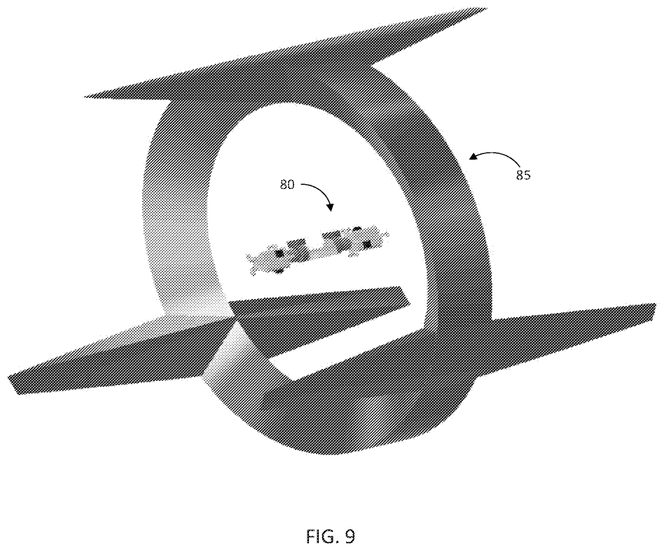

As illustrated in FIG. 9, the rings may be replaced by a cylinder 85 extending along a central axis. In such embodiments, the force actuator assembly/central axle 80, in combination with the cylinder 85, may be used for celestial navigation, satellite, and/or solar energy collection. The cylinder 85 can also provide for reflection of any frequency of electromagnetic radiation.

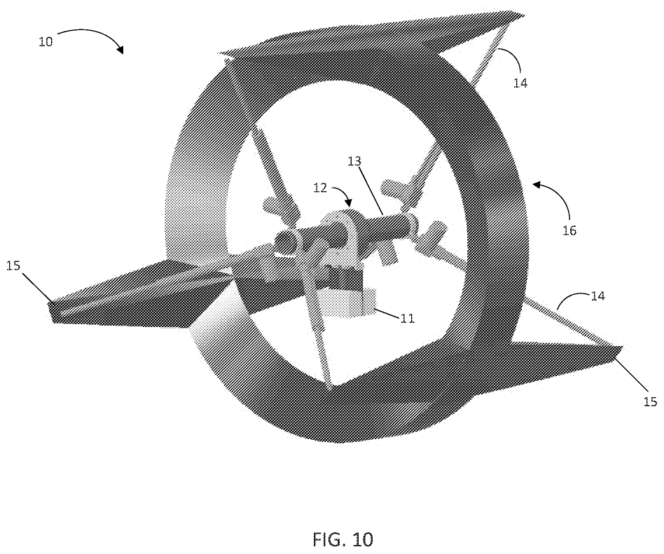

In another aspect, a tensegrity structure described herein comprises a hollow rotational motor attached to a central axis. A load is attached to the hollow motor and may be shifted along the axle axis either by movement of the motor assembly and/or load along the axle or may be shifted at an angle. FIG. 10 illustrates a perspective view of a tensegrity structure employing a hollow rotational motor assembly attached to a central axle, according to some embodiments. In the embodiment of FIG. 10, a load 11 is attached to the rotational motor assembly 12. A central axle 13 passes through the rotational motor assembly 12. Cables 14 extend from the ends of the central axle 13 to couple with struts 15 of the tensegrity structure 10. The struts 15 are coupled to a cylinder or rim 16.

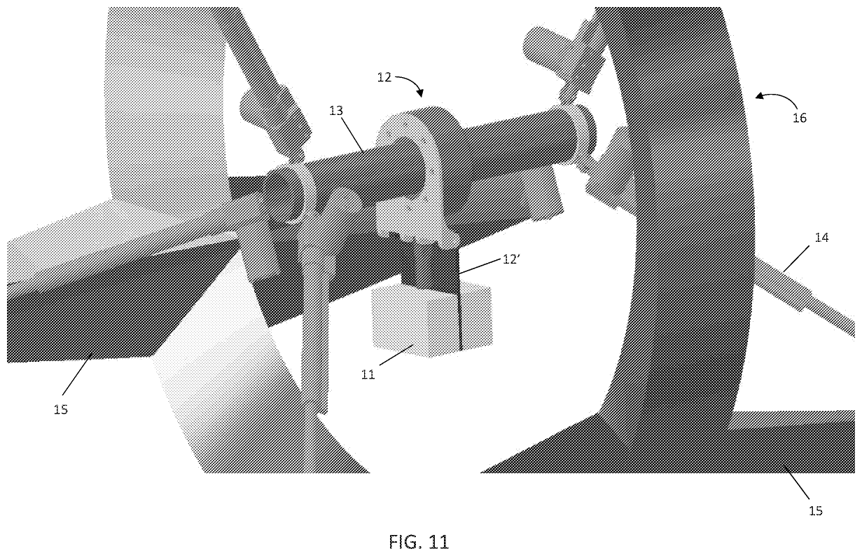

FIG. 11 is a magnified view of the tensegrity structure of FIG. 10. As illustrated in FIG. 11, a DC geared servomotor 12′ is attached to motor 12, wherein the DC geared servomotor 12′ provides lateral load shifting by tilting the load at an angle. This angular load shifting motor provides center-of-mass shifting laterally while the hollow motor adds to rotational motion of the rim 16. This simplification also does not require motors on the 6 cables (two 3 cable points), allowing for simple transport-only functions where aiming of axle is not required. By adding motors, sensors, controls, and other aspects of control, several other applications are possible including satellite optical communication, radio communication, position navigation and timing, and general aiming of the axle. A COTS tensegrity wheel with and without cable actuation has been developed for use with a tubular mortar system that provides to 100's of kilograms of load carrying capability at an optimized efficiency of rolling. The most basic tensegrity wheel can be kicked forward in case of complete electrical failure otherwise it will roll and navigate at command under power.

Tensegrity structures described herein, when employed as wheels, benefit from many modalities of motion within various fluids and atop solid surfaces. The transition between generating lift in air to rolling along a surface benefits from quick flipping of the tensegrity structure. The dual hollow rotational servos, over the single hollow servo, may provide advantage in gyroscopic maneuvering. Springs serving a force sensing through inductance measurement across the conductive surface of the spring may serve as additional compliant sensing for control of the tensegrity structure. Otherwise digital servo force control, sometime referred to as a digital spring may suffice to control tensions and positions.

Space applications for the tensegrity structure may also allow sampling, drilling, printing regulus, and inspection of objects of interest among other scientific analytical tasks. Attaching a laser range finder to the axle can provide 3D mapping of an environment of interest. The axle may also direct other imaging modalities such as ultrasound summed into 3D along its axle vector or radar. The struts may also serve as hoop antenna for radar applications while rotating or stationary. Within a zero-gravity environment, 3D printing applications involving a fiberoptic (FEP or other material) cable transmitting UV cross-linking radiation, much like a needle, coupled to the axle and inserted into a bag of UV solidifying fluid allows 3D printing. All modalities of 3D printing may be applied with the tensegrity wheel configuration in 1G or other acceleration environments.

Increases in rolling efficiency by tensegrity structures described herein support lift generation through rotation of a cylinder hollow body rim. This may be comprised of heat shield material and may rotate and generate lift. Additionally, a hollow ring rim may be used as a foil surface, such as a smoothed more tubular sheet metal version.

The rim can also encompass a fairing atop a rocket with central cargo on the axle. Also, within the application of rocketry, the central axle may be coupled to a thrust generating engine and provide thrust vectoring. Alternatively, engines such as solid-state booster rocket may be attached to the struts or other parts of the rim, and the thrust can be vectored by shifting the central axle mass through its workspace. This allows for thrust vectoring simply by shifting the central mass and subsequently the center of gravity. Additionally, the axle may be used for vectoring counter-rotating blades to create a thrust vector drone with canceling gyroscopic forces.

Principles of tensegrity structures and apparatus described herein are further described in the following non-limiting example of a wheel structure.

### Wheel Rim

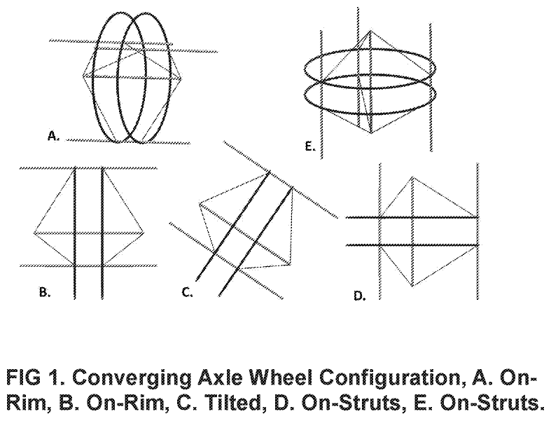

The rim of the wheel is minimally composed of two hoops axially offset to form a ring (FIG. 1) and attached together by three perpendicular struts. These struts are equidistantly placed around the rim and are of length approximately equal to the diameter of the hoop. A fixed distance axially separates these hoops equal to approximately twenty percent of the diameter of the hoop. This minimal rim structure (two hoops with three struts) provides support to the struts, keeping them perpendicular to the hoops, while also holding the hoops in place. Depending on materials and attachments, as the distance between the hoops is minimized or maximized, the support of the struts may decrease. The hoops are spaced equally from the centers of the struts at a width that increases lateral stability while in the rolling position and, subsequently, decreases the need for energy expenditure to maintain balance while rolling. However, too much lateral stability detrimentally inhibits positional changes.

The struts' lengths in this design are approximately equal to the diameter of the ring. These struts should not be so long that the wheel is prevented from tilting onto its side, i.e. into the standing position. The struts provide a lateral stability while the wheel is rolling, preventing unintended tilting, but also present a hazard to hitting nearby objects. These struts may be bent inward to minimize point contacts on impact and form a kind of ball for surface impact. To minimize the volume of the rim for launch or reentry, the struts and hoops may be twisted or folded such that the struts lie nearly parallel to the hoops, and the width of the ring is minimized. A folding ring demonstrates an efficient packing of this type of rim for launch.

In this design-build, the rim contains no sensors, electronics, or motors, mitigating the need to electrically connect the rim to the central axle. The only connections between the rim and axle are the cables providing actuation of the axle relative to the rim. Electrical connection between the axle and rim would be included if, for example, solar panels were located on the inner or outer surfaces of the ring, and thus power transfer to the axle would be necessary.

Modest rim flexibility may aid in positional changes by allowing the structure to bend into or out of positions, as well as facilitating turning while rolling by twisting the rim. The rim flexibility, in addition to easing transitions and absorbing impacts, may better conform to nonuniform surfaces and increase efficiency. Present rim designs do not attempt to optimize contact surfaces of the struts or ring for a surface environment, such as providing tread for granular materials. Optimization of this basic design into a lattice-type-structure of plastic or metal is not presented in this paper. To ease inducing a moment in the rim, the axle mass is maximized compared to the rim mass necessary to support and protect it.

### Wheel Axle

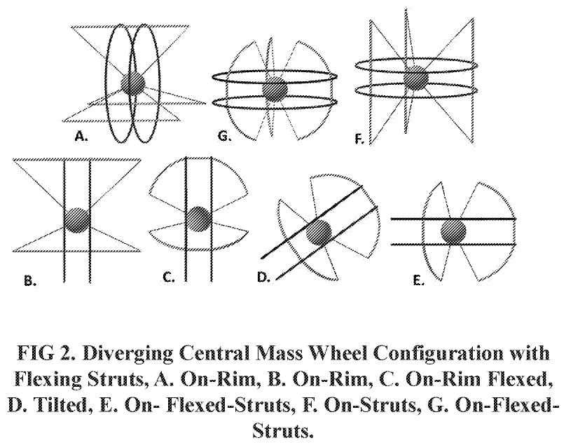

This axle's length may be longer or shorter than the cable attachment locations on the three struts. These two situations constitute the two design extremes presented here: converging long axle and diverging central mass wheels. In either design, there is a minimum of six cables which exit through the two axle ends (three cables at each end) and attach to the three struts of the rim in pairs (one from each end). The insertion locations of the cables along the struts may be more medial or distal to the axle ends. If the cable attachments on the rim are medial to the axle ends, it will be referred to here as converging (FIG. 1). If the cable insertions are distal along the struts, and the axle ends are medial, it will be referred to as diverging (FIG. 2).

The central axle contains all of the electronics, motors, sensors, batteries, and instruments. In this design-build, no traditional encoders on the cables or motors, absolute or incremental, are included. Instead, forces on the cables, both angular and tension, are used for locomotion and control. Because celestial bodies may have limited magnetic fields, the only orientation sensor is a 3D accelerometer located at the center of the axle. This 3D accelerometer will determine the gravity vector.

For simplicity, it is assumed the center-of-mass of the axle is balanced and central to the axle. The central axle may have a maximal length approaching the length of the struts or may minimally be a central mass (the extreme diverging case). Each of these axle situations provides advantages: the long axle version provides better pitch and yaw, while the central mass provides maximal protection from impact. Pitch and yaw may arguably be of less importance but could also provide better thrust vectoring of an axial engine, such as a cold gas thruster. Pitch and yaw may also be of benefit in applications such as laser satellite communication systems, or for directing an antenna. This yaw and pitch may also allow more nuanced analysis of objects of interest in either position.

The central axle must also be able to move radially toward the rim to such an extent that the axle's center-of-mass is able to adequately induce a moment and thus rotational motion around the rim. Increasing axle mass relative to the rim eases moment induction. Increasing the length of the axle limits the radial workspace within the rim due to the steep cable angles as the axle approaches the rim. This steep angle limits the radial force production of the cables.

The workspace of the axle's center-of-mass should, optimally, allow static moment induction in the rim, but axle momentum may also be used. This may be a less efficient or predictable method to produce motion between the axle and rim. This momentum method is more applicable to positional changes that might require overcoming stability where large excursions beyond the workspace of the axle would be required to statically induce lateral moments.

Depending on the axle length and cable insertion locations, the axial workspace may traverse well outside the volume of the rim. Both radially and axially, the workspace is limited by cable force production at steep angles. The central mass design allows an extreme range of movement of the axle's center-of-mass beyond the ring's volume but within the struts' volume. The long axle version allows the tips of the axle to extend axially beyond the volume of the wheel, but the center-of-mass workspace is muted by angular force limits. The central mass design's cable insertion locations are more distal on the struts to enable the center-of-mass to move axially past the hoops' edges. As shown in FIGS. 2C, 2D, 2E, and 2G, these distal strut insertion locations present the possibility of flexure of the struts, along with accompanying instability and motion-planning challenges.

Neither design, converging long axle and diverging central mass wheels, allows cables to cross and create interferences. Such cable interference designs may solve the need for large workspaces while providing increased pitch and yaw capabilities.

### Rotational Motion

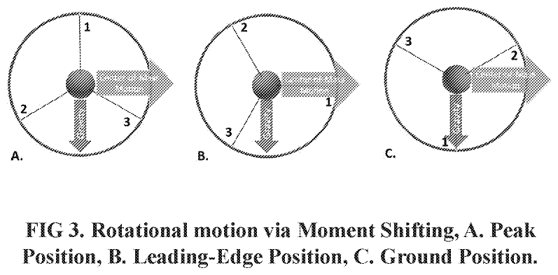

Rotational motion of either wheel design is dependent on inducing a moment in the rim by moving the axle radially and perpendicularly to the normal force on the rim (FIG. 3). FIG. 3A shows strut and cable set 1 in the apex position. From this apex position, the axle center-of-mass is moved radially toward the rim in the direction of motion, (rightward in this depiction) perpendicular to the gravity vector determined by the axle's accelerometers. As the wheel rotates to the right, the axle is continually moved perpendicular and to the right of the gravity vector. In this situation, and assuming a flat surface, the cable tensions in each pair of cables should be equivalent. From a control perspective, the absolute length of cable 1 would be minimized during all positions shown in FIG. 3. During these rotational phases, any cable in front of the axle in the direction of motion would be under positional feedback (minimizing length), while cables positioned behind the axle would be under force feedback control to maintain a desired tension. This would provide forward motion, while the reverse would decrease forward motion.

While rolling, there is a need to steer the wheel. While tensegrity wheel designs with dynamic rim radii may allow turning more easily by altering the left-to-right-hoop-radii ratio, the fixed rim radii presented here does not allow this type of motion. Instead this design relies on shifting the axle center-of-mass to the left or right of the rim's center-of-mass to provide left/right steering by slightly tilting the rim to one side. Position B in both FIGS. 1 and 2 shows the wheel through the sagittal plane between the hoops. In this B position, the axle would be shifted left or right, slightly tilting the rim to turn the wheel. In both cases, the sets of cable tensions would be offset to one side. Flexibility of the rim structure plays a role in allowing a bending, or twisting, of the rim instead of pure tilting on either hoop's edge.

Rather than measuring the more optimal rotation of the rim, due to the axle's limited pitch and yaw within the rim, monitoring the cycles of the axle (or rim) through the gravity vector during rolling motion could act as an odometer. While rolling down a negative incline, if energetics permit, dynamo power regeneration is also possible using the weight of the axle to pull out the apex cable while that same cable is reeled back in when not in the apex position. As kite power is generated by wind, this tensegrity wheel could generate power through the potential energy of the terrain.

### Positional Changes

Primary positional changes are between rolling and standing. During the rolling cycle, there exists an apex strut as shown in FIGS. 1B and 2B. While a strut is in the apex of the rim, it is optimally placed to use the weight of that strut, along with the center-of-mass of the axle, to induce a maximal lateral moment in the rim. As shown in FIGS. 1C and 2D, with one strut in this apex position, the other two lower struts are optimally placed around the circumference of the rim to provide a secondary pivot into the lateral tilt.

The first pivot for rolling-to-standing positional change is the edge of the lateral hoop on the rim's ring in the direction of motion. The second pivot-point is the line of the two non-apex strut ends on the side of the tilt. During tilting through both pivots, the axle's center-of-mass should be moved laterally past the pivots to induce the lateral moment. This moment induction approach may be augmented with momentum-based jerking motions. This lateral center-of-mass shifting is more easily accomplished with the diverging central mass design due to its larger workspace, containing more lateral cable insertions on the struts. If the cable insertions of the long axle design were brought together at the exact center of each strut, more lateral workspace would be possible.

Once in the standing position as shown in FIGS. 1D and 2E, the robot converts from the dynamic rolling orientation into a static precision inspection position. 3D representations are shown in FIGS. 1E, 2F and 2G. Any axial inspection instruments may be scanned over the surface of the celestial terrain under the standing robot or at angles toward nearby objects. Thrust vectoring or communications devices, as discussed above, may be precisely controlled in this stable, standing position.

In the standing position, the converging long axle design provides little to no control over the strut tip placements or flexion. There may exist a need to move the struts while in the standing position to locate the robot over an object of interest more precisely and without the need to change positions back from standing to rolling. This walking-type motion, using the struts as supporting legs, may be desired over repositioning the wheel, inspecting the object from the rolling position, and then moving back into a standing position.

For walking motion, the converging axle design allows the tip of the long axle to press against the surface. Assuming the weight of the rim could be lifted in the standing position, the long axle end would push into the ground, lifting the strut(s) and rim at least partially, then the axle would tilt and drag the rim and struts across the surface in the desired direction. As with drop impacts in the converging design, this walking presents challenges to protecting the ends of the axle from damage. An axle that is too short or insertion locations that decrease the downward force of the axle limit the ability of the axle to lift the rim. As the rim weight increases, this mode of walking becomes less feasible and efficient. This mode of walking would then only be suitable for minor adjustments in location and not a mode of gross locomotion.

Walking, for the diverging central mass design, is limited since the axle length is minimized, and there is no way to walk using the axle as a leg. Walking may, however, be undertaken by bending the struts to cause strut-end deflection. This provides flexion in the direction of the struts. However, any force generated by one strut is distributed through the cable network to the other struts. To address this, the off-center positioning of the central mass may help in providing non-uniform force distribution and directional walking-type-motion.

### Methodology

### Axle Build

FIG. 4A shows the axle build that was developed to validate and explore controls for the designs presented here. The main shaft of the axle is composed of a 3-foot long aluminum tube with 0.5-inch outer diameter. At the center of the axle, there is a single myRIO FPGA with 3D accelerometer for control of six L293D motor drivers and force sensing through twelve INA125 instrumentation amplifiers multiplexed through two MAX4619's. Each axle end is tipped with a 3-sided holder for the force sensors and motors as shown in FIG. 4B. Each cable is wound over two 17 mm ball bearing nylon pulleys attached to two m5 nylon clevis rod ends and two 20 kg load cells. Cables exit the tension sensor pulley with a static angle, while the cable exits the angular force sensor pulley with varying angles over the axle workspace.

FIG. 4C shows the 3D printed PETG motor and sensor holder with motors and sensors assembled. Each load cell is attached with a nylon bolt to a tapped hole in the 3D print. A second version was printed using carbon fiber nylon on a Robox 3D printer to increase durability (not shown). Each Dyneema cable is tensioned around 7 mm spools driven by micro-gear DC motors (maximums: 1.6 amp, 90 rpm, 70 oz-in). To protect each axle end, eight-inch diameter polyethylene floats were attached to each threaded axle rod end through two-inch sections of ⅜″-16 threaded rod. The total weight of the axle is approximately 2.5 kilograms, including float ends, all electronics, and a 12 amp-hour capacity 11.1-volt lithium polymer battery.

### PEX Tubing Rim Build

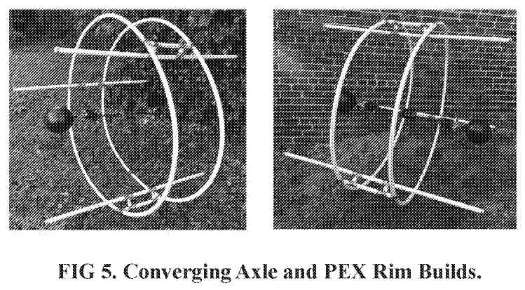

Each ring is constructed from three sections of five-foot sections of 1-inch diameter PEX tubing connected by three 1-inch brass tee connectors. All tubing is stainless-steel crimp-fitted to each tee connector. Three struts are also made of five-foot sections of PEX tubing. The approximate radius of the rim is 74 cm. FIG. 5 shows the axle in the PEX based rim. The PEX rings are connected roughly one-foot apart.

### Aluminum T-Bar Rim Build

Each ring is constructed from eight-foot sections of 6061 Aluminum T-Bar with 0.063-inch wall thickness by 1-inch high by 1.25-inch wide. Three T-bar sections make up each of the rim's two rings. The T-bars were bent along their height to an approximate radius of 116 centimeters using an electric tube bender. Each strut is constructed from an eight-foot section of 6063 Aluminum 90-degree angle with 0.063-inch wall thickness by 1.5-inch high by 1.5-inch wide. Attachment at the joint was aided by spanning the joint with two 4-inch long pieces of 90-degree angle with 0.063-inch thickness by 0.75-inch high by 0.75-inch. Additionally, a 6-inch long piece of 0.063-inch thick by 1.5-inch wide piece of aluminum spanned the inner width of the T-bar constructed ring and is also bolted perpendicularly to the struts.

### Polypropylene Tubing Rim Build

This build is made from polypropylene irrigation tubing with an outer diameter of approximately 0.7-inch. Tee connectors connect the tubing to form a circle of approximately 42 inches in diameter. The struts consist of three-foot sections of ⅜-inch aluminum rod. The rings are a maximum of 12 inches apart and a minimum of 5 inches apart.

### Procedures and Results

As shown in FIG. 4B, each cable runs through two force sensors: one measuring tension and one measuring the angular force of the exiting cable. With the motor and spool combination selected, the full force range of the sensors could, theoretically, be generated at a maximal unloaded speed of ˜3.3 cm/second. The motors were not fully stressed during these experiments so as to not damage them, however, no weaknesses in the structure were seen during the analysis of the angular force versus tension sensors.

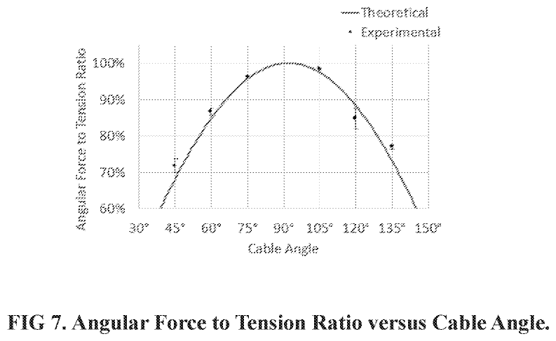

As a test of the angular force and tension sensors, known weights ranging from 300 grams to 4 kilograms were suspended by the cables at fixed angles ranging from perpendicular plus or minus 15 degrees, 30 degrees, and 45 degrees. As shown in FIG. 7, the calibrated tension and angular force were measured and compared at each of the chosen angles. The results were then curve fitted in Matlab with a sum-of-sines function with a resulting R-square of 0.9544. The polypropylene rim build weighed approximately 13.8 Newtons and had a rotational moment of inertia about its axis of ˜710 Ncm². The varying distance between the rings allows regions of greater lateral or radial stability. Positional transitions are made easier during phases of rotation where the width of the rim is minimized. This varying width may decrease the efficiency of rotation while providing phases of greater rotational stability or resistance to rotational motion.

The PEX rim build weighed approximately 40 Newtons and had an inertia of ˜2960 Ncm². The PEX rim was modified from FIG. 5 such that the weight was decreased, while rigidity increased through decreasing tee connections. Though the PEX material is not rigid enough to support large weights, it does withstand impacts and stresses without the deformation of the aluminum rim. The aluminum rim weighed approximately 57 Newtons and had a rotational moment of inertia of 6600 Ncm². The aluminum rim lacked necessary rigidity due, in part, to poor connections between the struts and the rings.

The simplified tensegrity wheel designs discussed here provide rotational motion in an efficient rolling configuration and walking motion in a stable tripodal configuration. The addition of angular force-to-tension ratio sensors for triangulation of the axle end position is useful as a direct localization technique. This force-only method of localization may be combined with force metrics for stress-strain analysis on the cable. This is in leu of absolute trilateration of each axle endpoint through cable encoders or other direct encoder sensors. Ultimately the goal is to produce an axle that may directly determine arbitrary, and possibly changing, cable attachment locations in the workspace. Cable length, direction, tension, and angular force may all then contribute to defining a configuration space for the axle within varying cable attachment locations.

In future studies, absolute cable length will be calibrated with gimbaled pull-wire potentiometers affixed onto each axle end at the cable attachments. Other distance calibration sensors may include incremental indexed encoders on each cable motor, optimized amplifiers on each force sensor, and a vision system. These may enable this wheel system to gain greater autonomy in its environment. To better protect the cable system and force sensors, they will be moved more centrally, while the angular force sensor system will be integrated into the ends of the axle or replaced by directional sensors. Onboard Kalman filtered IMU or external vision system will be used for motion capture of the wheel during drop simulations and movement tests. While extremely preliminary, this wheel design rolls down uneven terrain very well and, with the right materials and geometry, may prove well-suited for the extreme environments of celestial body exploration.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A tensegrity structure comprising:
a plurality of rings spaced apart along a central axis and connected by struts, wherein the struts are coupled to a force actuator assembly via cables, the force actuator assembly residing within a volume defined by the struts.

2. The tensegrity structure of claim 1, wherein three struts connect two rings.

3. The tensegrity structure of claim 1, wherein the cables extend from opposing ends of the force actuator assembly.

4. The tensegrity structure of claim 1, wherein the force actuator assembly resides within a volume defined by the rings.

5. The tensegrity structure of claim 1, wherein the force actuator assembly comprises angular force and tension sensors.

6. The tensegrity structure of claim 1, wherein the force actuator assembly comprises at least one servo driven slip ring, the slip ring coupled to one or more weights.

7. The tensegrity structure of claim 6, wherein the one or more weights comprise batteries for running one or more components of the force actuator assembly.

8. The tensegrity structure of claim 6, wherein the force actuator assembly comprises at least two servo driven slip rings.

9. The tensegrity structure of claim 8, wherein the two servo driven slip rings are operable to rotate independently of one another.

10. The tensegrity structure of claim 1, wherein the force actuator assembly comprises a single hollow rotating motor and a central axle passing through the rotating motor.

11. The tensegrity structure of claim 10, wherein one or more weights are coupled to the rotating motor.

12. The tensegrity structure of claim 10, wherein the force actuator assembly does not comprise motors coupled to the cables.

13. The tensegrity structure of claim 1, wherein the tensegrity structure functions as a wheel.

14. A tensegrity structure comprising:
a cylinder extending along a central axis and connected to a plurality of struts, wherein the struts are coupled to force actuator assembly via cables, the force actuator assembly residing within a volume defined by the struts.

15. The tensegrity structure of claim 14, wherein the cables extend from opposing ends of the force actuator assembly.

16. The tensegrity structure of claim 14, wherein the force actuator assembly comprises at least one servo driven slip ring, the slip ring coupled to one or more weights.

17. The tensegrity structure of claim 16, wherein the force actuator assembly comprises at least two servo driven slip rings.

18. The tensegrity structure of claim 17, wherein the two servo driven slip rings are operable to rotate independently of one another.

19. The tensegrity structure of claim 14, wherein the force actuator assembly comprises a single hollow rotating motor and a central axle passing through the rotating motor.

20. The tensegrity structure of claim 19, wherein the force actuator assembly does not comprise motors coupled to the cables.

21. The tensegrity structure of claim 14, wherein the cylinder comprises one or more photovoltaic sections.

22. The tensegrity structure of claim 14, wherein the cylinder assists in receiving and/or transmitting electromagnetic radiation.

23. The tensegrity structure of claim 14, wherein the tensegrity structure is a satellite or thrust vectoring apparatus.

24. An apparatus for studying human motion comprising:
a tensegrity structure of claim 1 or claim 14 coupled to a model of a human body or a model of any portion of a human body.

25. The apparatus of claim 24, wherein the model comprises a human torso for studying running and/or walking motions.
